(19) United States
(12) Patent Application Publication
(10) Pub. No.: US 2026/0308092 A1
(43) Pub. Date: Oct. 8, 2026

(54) PROCESS FOR PREPARING 2,3-UNSATURATED ALCOHOLS

(71) Applicant: BASF SE, Ludwigshafen am Rhein (DE)

(72) Inventors: Sebastian WLOCH, Ludwigshafen am Rhein (DE); Christoph STOCK, Ludwigshafen am Rhein (DE); Jonathan Thomas LEFEBVRE, Ludwigshafen am Rhein (DE); Margarethe KLOS, Ludwigshafen am Rhein (DE); Bernhard BRUNNER, Ludwigshafen am Rhein (DE)

(21) Appl. No.: 19/482,266

(22) PCT Filed: May 3, 2024
(86) PCT No.: PCT/EP2024/062302

§ 371 Date: Nov. 7, 2025

(30) Foreign Application Priority Data
May 10, 2023 (EP) 23172557.3

(51) Int. Cl. C07C 29/141; B01J 23/89; B01J 31/18

(52) U.S. Cl. CPC C07C 29/141; B01J 23/8946; B01J 31/181; B01J 2231/52; B01J 2531/004; B01J 2531/66

(57) ABSTRACT

The present invention relates to a process for the preparation of unsaturated alcohols of the formula

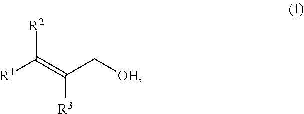

which comprises subjecting unsaturated aldehydes of the formula

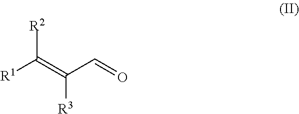

to a hydrogenation in the presence of a catalyst and a tertiary amine; characterized in that the hydrogenation takes place in a reactor cascade. Conducting the hydrogenation in a reactor cascade, in particular a cascade of stirred vessel reactors, a cascade of bubble column reactors, or a cascade of jet loop reactors, reduces the amount of formed side products and enhances yield and selectivity of the products.

The present invention relates to a process for the preparation of unsaturated alcohols of the formula (I), which comprises subjecting unsaturated aldehydes of the formula (II) to a hydrogenation in the presence of a catalyst and a tertiary amine; characterized in that the hydrogenation takes place in a reactor cascade. Conducting the hydrogenation in a reactor cascade, in particular a cascade of stirred vessel reactors, a cascade of bubble column reactors, or a cascade of jet loop reactors, reduces the amount of formed side products and enhances yield and selectivity of the products.

### PRIOR ART

U.S. Pat. No. 6,916,964B2 (EP1318128A2) relates to a process for selective hydrogenation of α,β-unsaturated carbonyl compounds of the general formula I

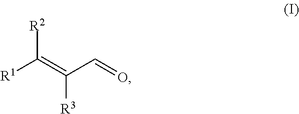

where
R¹ and R² are identical or different and are each independently hydrogen or a singly or multiply unsaturated straight chain or branched substituted or unsubstituted C₁-C₂₀-alkyl radical, an unsubstituted or substituted aryl radical or an unsubstituted or substituted heterocyclic group, and
R³ is hydrogen or C₁-C₄-alkyl, together with corresponding unsaturated alcohols of general formula II

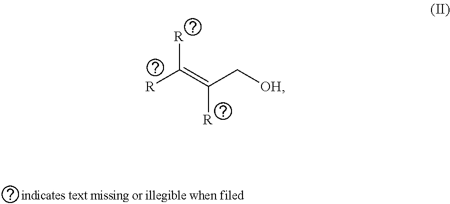

where R¹, R² and R³ are each as defined above, in a reactor containing, in the absence or presence of a gas phase, a liquid phase holding at least one catalyst in suspension, which comprises passing the liquid phase and, if present, the gas phase through a device in the reactor having openings or channels whose hydraulic diameter is in the range from 0.5 to 20 mm.

The hydrogenation is carried out in a reactor according to EP798039, in which liquid and gas phases are passed through a device having openings or channels having a hydraulic diameter in the range from 0.5 to 20 mm, preferably 1 to 10 mm, particularly preferably 1 to 3 mm. The hydraulic diameter is defined as the quotient of 4 times the cross-sectional area of the opening and the circumference thereof. The hydrogenation can be carried out in various reactor forms, such as jet nozzle reactors, bubble columns or multitube reactors. According to Example 1 the hydrogenation was carried out in a bubble column (3000 mm in length, 27.3 mm in diameter) equipped with a fabric packing.

U.S. Pat. No. 7,101,824B2 (EP1317959B1) relates to a process for preparing a ruthenium/iron, catalyst supported on carbon, comprising besides 0.1 to 10% by weight of ruthenium on a carbon support 0.1 to 5% by weight of iron, by:
a) suspending said support in water,
b) simultaneously adding the catalytically active components ruthenium and iron in the form of solutions of their metal salts,
c) simultaneously precipitating the catalytically active components onto the support by addition of a base,
d) separating the catalyst from the aqueous phase of the support suspension,
e) drying the catalyst,
f) reducing the catalyst in a hydrogen stream at from 400 to 600° C., and
g) conditioning the catalyst under low flammability liquids or passivating the catalyst with a dilute oxygen stream; and to a process for the selective liquid phase hydrogenation of carbonyl compounds of the general formula

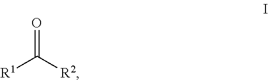

where
R¹ and R² are identical or different and are each independently hydrogen or a saturated or mono- or polyunsaturated straight chain or branched substituted or unsubstituted C₁-C₂₀-alkyl radical, an unsubstituted or substituted aryl radical or an unsubstituted or substituted heterocyclic group, such as, for example, citral, said process comprising the step of reducing said carbonyl compounds, in the presence of the catalyst prepared by the above described process, into corresponding unsaturated alcohols of the general formula

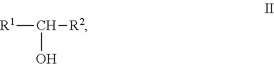

where R¹ and R² are each as defined above.

The hydrogenation process can be carried out either continuously or batchwise in suspension or in a fixed bed. The continuous process is particularly advantageous. The continuous or batchwise suspension process can be carried out as described, for example, in EP947943 (U.S. Pat. No. 6,369,277B1) and U.S. Pat. No. 5,939,589 respectively.

U.S. Pat. No. 6,369,277B1 (EP947493) relates to a process of selectively hydrogenating an α,β-unsaturated carbonyl compound of the formula (I)

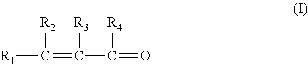

where R₁ is saturated C₁₄₀-hydrocarbyl, unsaturated hydrocarbyl or a substituted or unsubstituted aromatic radical containing moiety, and R₂, R₃ and R₄, independently of one another, are hydrogen or a C₁- to C₄-alkyl group, in the liquid phase to a saturated carbonyl compound of the formula (II)

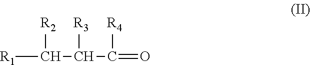

with hydrogen in the presence of a pulverulent palladium and/or rhodium catalyst and in the presence of an organic base, which comprises: conducting the hydrogenation in a packed bubble column reactor in which product is recycled and hydrogen gas is recirculated.

EP0071787A2 relates to ruthenium-on-charcoal and ruthenium-on-carbon black hydrogenation catalysts, their preparation and their use for selective hydrogenation of unsaturated carbonyl compounds.

DE10223974 relates to a process for the continuous isolation of two stereoisomeric isoprenoid alcohols from a crude mixture by rectification.

WO2004007414 relates to a method for the continuous hydrogenation of citral to give citronellal is disclosed, whereby a liquid phase, in which citral is dissolved and the particles of a catalyst, for the preferential hydrogenation of carbon-carbon double bonds before carbon-oxygen double bonds, are suspended, is fed through a device in the presence of a gas comprising hydrogen, said device preventing the transport of the catalyst particles.

WO2007/045641A1 relates to a method for the continuous hydrogenation of unsaturated compounds, according to which particles of a first hydrogenating catalyst are suspended in a liquid phase in which an unsaturated compound is dissolved. The liquid phase is guided through a packed bubble column, in the presence of a hydrogenous gas which is subjected to a first partial hydrogen pressure and is at a first temperature in the parallel flow in the opposite direction to the gravitational force, the substance discharged from the bubble column reactor is subjected to a gas-liquid separation, and the liquid phase is then subjected to a transversal filtration, whereby a retentate and a filtrate are obtained. The retentate is introduced back into the bubble reactor, and the filtrate is guided over a bed of a second hydrogenating catalyst in the presence of a hydrogenous gas subjected to a second hydrogen partial pressure and at a second temperature, the second hydrogen partial pressure being at least 10 bar higher than the first hydrogen partial pressure.

U.S. Pat. No. 5,939,589 relates to a process for carrying out catalytic reactions in a reactor which contains a liquid phase in which at least one catalyst is suspended, and which may additionally contain a gas phase, wherein the liquid phase and, where relevant, the gas phase are fed, at least partly, through an apparatus having orifices or channels in the reactor, the hydraulic diameter of which orifices or channels is from 0.5 to 20 mm, preferably from 1 to 10 mm, particularly preferably from 1 to 3 mm, wherein said orifices or channels have wall materials which have surface roughnesses of from 0.1 to 10, preferably from 0.5 to 5, times the mean particle size of the suspended catalyst particles, and wherein the liquid phase is fed through the apparatus having orifices or channels at an empty tube velocity of from 50 to 300, preferably from 150 to 200, m³/M² h.

WO2017/211784A1 relates to a process for the preparation of an unsaturated alcohol of the formula

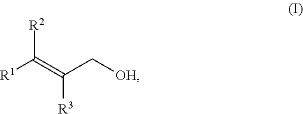

wherein
R¹ and R² are each independently from one another selected from hydrogen, C₁-C₂₀-alkyl which is unsubstituted or substituted with 1 to 5 identical or different radicals R⁴, C₂-C₂₀-alkenyl which contains 1, 2, 3, 4 or 5 double bonds and is unsubstituted or substituted with 1 to 5 identical or different radicals R⁵, aryl which is unsubstituted or substituted with 1 to 5 identical or different radicals R⁶, and 3-, 4-, 5-, 6- or 7-membered saturated, partially unsaturated or aromatic heterocyclic rings containing as ring members 1, 2 or 3 heteroatoms which are, independently of each other, selected from N, NR^{a}, O and S, wherein the heterocyclic rings are unsubstituted or substituted with 1 to 5 identical or different radicals R⁶; provided that R¹ and R² are different from each other; R³ is selected from hydrogen and C₁-C₄-alkyl;
R⁴ is selected from hydroxyl, cyano, nitro, halogen, C₃-C₇-cycloalkyl, C₁-C₄-alkoxy, C₁-C₄-alkoxycarbonyl, C₁-C₄-alkylcarbonyloxy, —NR^{7a}R^{7b} and aryl which may be unsubstituted or substituted with 1 to 5 identical or different radicals R⁵;
R⁵ is selected from hydroxyl, cyano, nitro, halogen, C₁-C₄-alkyl, C₃-C₇-cycloalkyl, C₁-C₄-alkoxy, C₁-C₄-alkoxycarbonyl, C₁-C₄-alkylcarbonyloxy, —NR^{7a}R^{7b} and aryl which may be unsubstituted or substituted with 1 to 5 identical or different radicals R;
R⁵ is selected from cyano, nitro, halogen, C₁-C₄-alkyl, C₃-C₇-cycloalkyl, C₁-C₄-alkoxy and —NR^{7a}R^{7b};
R^{7a} and R^{7b} are each independently from one another selected from hydrogen, C₁-C₆-alkyl, C₃-C₇cycloalkyl, C₁-C₆-alkoxy, C₂-C₆-alkenyl, C(═O)R⁶, C(═O)OR⁹, phenyl and benzyl; or
R^{7a} and R^{7b} together represent a C₂-C₆-alkylene chain which in combination with the nitrogen atom it is bonded to forms a 3-, 4-, 5-, 6-, 7- or 8-membered saturated, partly unsaturated or aromatic ring, wherein the alkylene chain may contain 1 or 2 heteroatoms which are, independently of each other, selected from O, S and NR^{b}, and where the alkylene chain may optionally be substituted with 1, 2, 3 or 4 identical or different radicals selected from halogen, C₁-C₅-alkyl, C₃-C₇-cycloalkyl and C₁-C₆-alkoxy;
R⁸ is selected from hydrogen, C₁-C₆-alkyl, C₂-C₆-alkenyl, C₃-C₇-cycloalkyl, wherein the three last mentioned radicals may be unsubstituted or substituted with 1 or 2 identical or different radicals selected from hydroxyl and C₁-C₄-alkoxy;
R⁹ is selected from C₁-C₆-alkyl, C₂-C₆-alkenyl, C₃-C₇-cycloalkyl, wherein the three last mentioned radicals may be unsubstituted or substituted with 1 or 2 identical or different radicals selected from hydroxyl and C₁-C₄-alkoxy;
R^{a} is selected from C₁-C₄-alkyl, C₃-C₇-cycloalkyl and C₁-C₄-alkoxy;
R^{b} is selected from hydrogen, C₁-C₄-alkyl, C₃-C₇-cycloalkyl and C₁-C₄-alkoxy; which comprises subjecting an educt composition including at least 75% by weight of an unsaturated aldehyde of the formula (II)

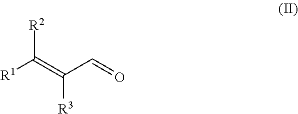

wherein R¹, R² and R³ have the above defined meanings, to a hydrogenation in the presence of a catalyst and a tertiary amine; wherein the tertiary amine is used in an amount ranging from 0.001 to 0.7% by weight, based on the total amount of the liquid reaction mixture.

US20180297013A1 (WO2017060243A1, U.S. Ser. No. 10/562,009) relates to a process for producing a ruthenium-iron-carbon support catalyst comprising from 0.1 to 5% by weight of iron in addition to from 0.1 to 10% by weight of ruthenium on a carbon support by
a) introducing a support into water
b) simultaneously adding the catalytically active components ruthenium and iron in the form of solutions of their metal salts
c) coprecipitating the catalytically active components on the support by addition of a base
d) separating the catalyst from the aqueous phase of the support suspension
e) drying the catalyst
f) reducing the catalyst in a stream of hydrogen at less than 400° C.
g) removing the catalyst from the reduction reactor under relatively nonflammable liquids or passivating the catalyst by passing a diluted oxygen stream over it or passivating the catalyst by passing a diluted oxygen stream over it and removal of the catalyst from the reduction reactor under relatively nonflammable liquids.

CN110963888A relates to a method for preparing nerol and geraniol from citral. The citral serving as a raw material undergoes a hydrogenation reaction under the action of a catalyst to generate nerol and geraniol; and the catalyst comprises an MOFs material (metal-organic framework compound) and a passivation component Si element, and the Si element exists in the form of an oxide.

Known catalytic hydrogenations of α,β-unsaturated carbonyl compounds, such as in particular citral, yielding the respective unsaturated alcohols, such as in particular mixtures of nerol and geraniol, suffer from insufficient selectivity. They therefore result in high levels of unwanted side products arising, e.g., from over-hydrogenation or olefin isomerization. The processes of the prior art are particularly disadvantageous as they lead to substantial amounts of several side products having boiling points closely to that of geraniol/nerol, which makes it difficult to fully separate them from the intended product geraniol/nerol by fractional distillation.

These side products are mainly citronellol on the one hand and the isonerol isomers on the other hand, which occur as side reactions during the hydrogenation reaction. At high conversion rates of citral to geraniol/nerol also the formation of citronellol is increased.

It was surprisingly found that conducting the hydrogenation of citral to give a mixture of geraniol and nerol in a reactor cascade, in particular a cascade of stirred vessel reactors, a cascade of bubble column reactors, or a cascade of jet loop reactors,
reduces the formed citronellol content relatively to the residual citral content;
enhances yield and selectivity of the process and, thus, less citral is needed to produce the desired amount of geraniol/nerol and less effort is needed to separate geraniol/nerol from citronellol to obtain the desired product purity.

Lower amounts of citronellol are advantageous, for example, in the isomerization of geraniol/nerol to linalool, because citronellol in the feed is reducing the available reactor volume and furthermore makes the separation of linalool from the educt mixture by distillation more difficult.

Accordingly, the present invention relates to a process for the preparation of unsaturated alcohols of the formula

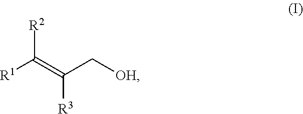

wherein
R¹ and R² are each independently from one another selected from C₁-C₂₀-alkyl which is unsubstituted or substituted with 1 to 5 identical or different radicals R⁴, C₂-C₂₀-alkenyl which contains 1, 2, 3, 4 or 5 double bonds and is unsubstituted or substituted with 1 to 5 identical or different radicals R⁵; provided that R¹ and R² are different from each other;
R³ is selected from hydrogen and C₁-C₄-alkyl;
R⁴ is selected from hydroxyl, cyano, C₃-C₇-cycloalkyl and C₁-C₄-alkoxy,
R⁵ is selected from hydroxyl, cyano, C₁-C₄-alkyl, C₃-C₇-cycloalkyl, C₁-C₄-alkoxy; which comprises subjecting unsaturated aldehydes of the formula

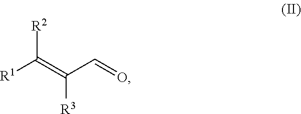

wherein R¹, R² and R³ have the above defined meanings, to a hydrogenation in the presence of a catalyst and a tertiary amine; characterized in that the hydrogenation takes place in a reactor cascade.

Conducting the hydrogenation in a reactor cascade, in particular a cascade of stirred vessel reactors, a cascade of bubble column reactors, or a cascade of jet loop reactors, reduces the amount of formed side products and enhances yield and selectivity of the products. An example of a jet loop reactor is a buss loop reactor. Accordingly, a cascade of buss loop reactors represents a specific embodiment of a cascade of jet loop reactors. In addition, a reactor cascade may also comprise a reactor cascade, which consists of two, or more different reactors, which are, for example, selected from a stirred vessel reactor, a bubble column reactor and a jet loop reactor.

The process of the present invention is preferably used for the preparation of unsaturated alcohols of the formula

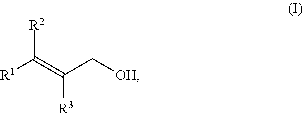

wherein one of R¹ and R² is C₄-C₁₀-alkenyl containing one double bond and the other one is methyl, or ethyl, especially methyl; and R³ is hydrogen.

Advantageously, the unsaturated aldehyde of the formula (II) is Citral, which is hydrogenated to give a mixture of geraniol and nerol.

The term “Citral” as used herein refers to a composition consisting largely, i.e. to an extent of usually at least 90% by weight, preferably at least 95% by weight and in particular at least 98% by weight, of a mixture of neral and geranial in any proportion.

The term “C₁-C₂₀-alkyl” as used herein refers to a branched or unbranched saturated hydrocarbon group having 1 to 20, for example methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, heptyl, octyl, 2-ethylhexyl, nonyl and decyl and their isomers. C₁-C₄-alkyl means for example methyl, ethyl, propyl, 1-methylethyl, butyl, 1-methylpropyl, 2-methylpropyl, or 1,1-dimethylethyl.

C₁-C₄-alkoxy is, for example, methoxy, ethoxy, n-propoxy, 1-methylethoxy (isopropoxy), butoxy, 1-methylpropoxy (sec-butoxy), 2-methylpropoxy (isobutoxy) or 1,1-dimethylethoxy (tert-butoxy).

The term “C₂-C₂₀-alkenyl which contains 1, 2, 3, 4 or 5 double bonds” as used herein denotes a linear or branched ethylenically unsaturated hydrocarbon group having 2 to 20 carbon atoms and 1 to up to 5 C-double bonds wherein each of the carbon atoms is involved in not more than 1 double bond. C₂-C₂₀-alkenyl containing one double bond is for example, ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 11-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl, C₂-C₂₀-alkenyl containing two double bonds is for example 1,3-butadienyl, 1,4-pentadienyl, 1,3-pentadienyl, 2,4-pentadienyl, 2-methyl-1,3-butadienyl, 1,5-hexadienyl, 1,4-hexadienyl, 1,3-hexadienyl, 2,5-hexadienyl, 3,5-hexadienyl, 2,4-hexadienyl, 2-methyl-1,3-pentadienyl or 4-methyl-1,3-pentadienyl, C₂-C₂₀-alkenyl containing three double bonds is for example 1,3,5-hexatrienyl, 1,3,6-heptatrienyl, 1,4,7-octatrienyl or 2-methyl-1,3,5-hexatrienyl, C₂-C₂₀-alkenyl containing four double bonds is for example 1,3,5,7-octatetraenyl, 1,3,5,8-nonatetraenyl, 1,4,7,10-undecatetraenyl or 2-ethyl-1,3,6,8-nonatetraenyl, and C₂-C₂₀-alkenyl containing five double bonds is for example 1,3,5,7,9-decapentaenyl, 1,4,6,8,10-undecapentaenyl, 1,4,6,9,11-dodecapentaenyl or 2-ethenyl-1,3,5,8-nonatetraenyl.

The term “C₃-C₇-cycloalkyl” as used herein refers to a monocyclic and bicyclic 3- to 7-membered saturated cycloaliphatic radical, e.g. cyclopropyl, cyclobutyl, cyclopentyl, cyclohexyl, cycloheptyl, bicyclo[2.2.1]heptyl and bicyclo[3.1.1]heptyl. Preferably, the term cycloalkyl denotes a monocyclic saturated hydrocarbon radical.

### Catalyst

In a preferred embodiment of the invention the heterogeneous catalyst, as preferably used in the process according to the invention, is a supported catalyst comprising ruthenium that is supported on an inert support material. According to a particularly preferred aspect of this embodiment the supported catalyst additionally comprises a further transition metal that is also supported on the support material. The further transition metal is preferably iron. In addition, the ruthenium content of the supported catalyst is preferably in the range of 0.1 to 10% by weight, in particular of 1 to 7% by weight, while its iron content, if iron is included in the catalyst, is preferably in the range of 0.1 to 5% by weight, in particular of 0.1 to 3% by weight, based in each case on the dry weight of the catalyst. The supported catalyst can be used in the form of a powder. In general, such a powder has particle sizes in the range from 1 to 200 μm, in particular 1 to 100 μm. In a particularly preferred embodiment of the invention the catalyst used in the process according to the invention comprises both ruthenium and iron as supported on a support material which is a carbon carrier, in particular activated carbon. Such a catalyst is typically used in powder form. The catalysts that comprise one or more transition metals can be prepared by customary processes well-known in the art. The herein preferred supported catalysts comprising ruthenium and optionally iron are preferably obtained by the process disclosed in EP1317959A1 and especially by the process disclosed in WO2017/060243A1. Both documents are incorporated herein by reference.

In summary said processes comprising the following steps:
a) suspending the support material in water,
b) simultaneously adding ruthenium and optionally iron in the form of solutions of their metal salts,
c) precipitating ruthenium and, if applicable, iron essentially in the form of their hydroxides onto the support by the addition of a base,
d) separating the loaded support from the aqueous phase of the suspension,
e) drying the loaded support,
f) subjecting the loaded support to hydrogenation conditions at a temperature of below 600° C., and
g) conditioning the obtained catalyst under a liquid having low flammability, or passivating the obtained catalyst with a dilute oxygen stream, or passivating the obtained catalyst with a dilute oxygen stream and conditioning the catalyst under a liquid having low flammability.

The support material employed in step a) is typically in a finely divided form, such as for example powdered activated carbon. The aqueous suspension obtained in step a) may be used in step b) as is or may possibly be adjusted to a pH value below 7, in particular below 6, by adding an acid, e.g. nitric acid, or to a pH value above 7, in particular above 8, by adding a base, such as sodium hydroxide.

In step b) the addition of the ruthenium salt and, if applicable, the iron salt is preferably carried out at an elevated suspension temperature, in particular at a temperature of 50 to 95° C., and specifically at a temperature of 70 to 90° C.

Suitable salts of ruthenium and iron include their chlorides, nitrates, nitrosylnitrates, acetates, oxides, hydroxides and acetylacetonates, such as in particular ruthenium chloride, ruthenium nitrosyl nitrate, iron chloride and iron nitrate.

In step c) the base, such as for example sodium carbonate, sodium hydrogencarbonate, ammonium carbonate, ammonia, urea, sodium hydroxide, potassium hydroxide or lithium hydroxide, in particular sodium hydroxide, is typically slowly added to the suspension obtained in step b) until a pH value in the range of 6 to 14, preferably in the range of 8 to 12 and in particular in the range of 8 to 10 is reached. The base is preferably added to the suspension having an elevated temperature, in particular a temperature of 50 to 95° C., and specifically a temperature of 70 to 90° C. In step d) the loaded support is preferably separated by filtration and the obtained filter cake is then dried in step e) usually under reduced pressure or an inert gas atmosphere. In step f) the material obtained in step e) is reduced in a hydrogen stream, possibly diluted with an inert gas, such as nitrogen. The hydrogen content of the hydrogen stream is typically in the range of 5 to 100% by volume, preferably 5 to 50% by volume and in particular 5 to 25% by volume.

The reduction in step f) is preferably carried out at a temperature in the range of 100 to below 600° C., preferably 150 to below 550° C., and in particular 180 to 520° C. In one embodiment of the invention the reduction in step f) is carried out at a temperature in the range of 400 to below 600° C., preferably 450 to 550° C., and in particular 480 to 520° C.

In a preferred embodiment of the invention the reduction in step f) is carried out at a temperature of 100 to 400° C., preferably 120 to 300, in particular 150 to 250° C., especially 180 to 220° C., and specifically 190 to 210° C.

In step g) the catalyst obtained in step f) may be conditioned under a low flammability liquid, such as water, typically after cooling it to temperatures below 40° C. Alternatively or in addition to such a conditioning a passivation process can be carried out in step f). According to one embodiment of the present invention passivation is effected by means of a diluted oxygen stream usually at a temperature in the range of 10 to 30° C., such as ambient temperature. Preferred diluted oxygen streams have an oxygen content of below 50% by volume, in particular below 25% by volume, particularly preferred below 10% by volume, especially below 5% by volume and specifically of about 1% by volume. For diluting oxygen typically an inert gas, such as nitrogen, helium, neon, argon or carbon dioxide, and preferably nitrogen, is used.

### Tertiary Amines

Preferred tertiary amines have a boiling point substantially lower than or higher than, preferably substantially lower than the boiling point of nerol, and particularly preferred ones have a boiling point of less than 180° C. at a pressure of 1 bar. The tertiary amine may be selected from those disclosed in EP0071787. According to a preferred embodiment of the present invention the tertiary amine is selected from tri(C₁-C₂₀-alkyl)amines, more preferably from tri(C₁-C₅-alkyl)amines, in particular from tri(C₁-C₃-alkyl)amines, wherein the amine may comprise three identical alkyl groups, two identical alkyl groups, or three different alkyl groups; such as trimethylamine, triethylamine or N,N-dimethylethylamine, and specifically is trimethylamine.

The hydrogenation reaction of the inventive process may or may not be conducted in the presence of a solvent. Preferably the hydrogenation is carried out in the presence of such a solvent, in particular a protic organic solvent. In a preferred embodiment of the present invention the organic solvent is selected from C₁-C₈-alkanoles, in particular from methanol, ethanol and isopropanol, and specifically is methanol.

The solvent is typically added in an amount of 0 to 50% by weight, preferably in an amount of 5 to 40% by weight, in particular in an amount of 10 to 30% by weight and specifically in an amount of 15 to 30% by weight, relative in each case to the total weight of the liquid reaction mixture.

The hydrogenation is typically carried at a hydrogen pressure in the range from 1 to 100 bar, preferably in the range from 1 to 80 bar, more preferably in the range from 20 to 60 bar.

The hydrogen used for the hydrogenation can be used in pure form or, if desired, also in the form of mixtures with other, preferably inert gases, such as nitrogen or argon. Preference is given to using hydrogen in undiluted form.

The hydrogenation is typically carried at a temperature in the range from 40 to 120° C., preferably in the range from 60 to 100° C., more preferably in the range from 70 to 90° C.

In the process of the invention, for example, Citral is not used in pure form, but as an aminic alcoholic solution. Suitable alcohols are preferably C₁-C₄-alcohols, suitable amine components are preferably tertiary C₁-C₄-amines. The concentration of Citral in the solution is preferably in the range from 50 to 90% by weight, particularly preferably 60 to 80% by weight, the concentration of alcohol is in the range from 40 to 5% by weight, preferably 10 to 30% by weight, and the concentration of tertiary amine is in the range from 1.0 to 15% by weight, preferably 1.0 to 8% by weight, more preferably 1.0 to 5.0% by weight; based on the total amount of Citral, alcohol and amine (EP1317959).

In another preferred embodiment the tertiary amine is used in an amount ranging from 0.001 to 0.7% by weight, especially 0.005 to 0.5% by weight and very especially 0.01 to 0.2% by weight based on the total amount of neral (and geranial), alcohol and amine in the catalytic hydrogenation of neral (WO2017/211784A1).

The product obtained after hydrogenation of Citral is a mixture of geraniol and nerol having a level of the by-product citronellol in total of less than 2.0% by weight, preferably less than 1.6% by weight, and in particular from 0.3 to 1.0% by weight; in particular a mixture of geraniol and nerol, which comprises 88 to 97% by weight, preferably 90 to 96% by weight, of geraniol and nerol; and 2.0 to 0.1% by weight, preferably 1.6 to 0.3% by weight, more preferably 1.0 to 0.3% by weight of the by-product citronellol, relative in each case to the total weight of the geraniol/nerol compound.

### Reactor

The term “reactor cascade” means according to the present invention a series of N reactors which are arranged spatially separated from each other, i.e. are connected in series.

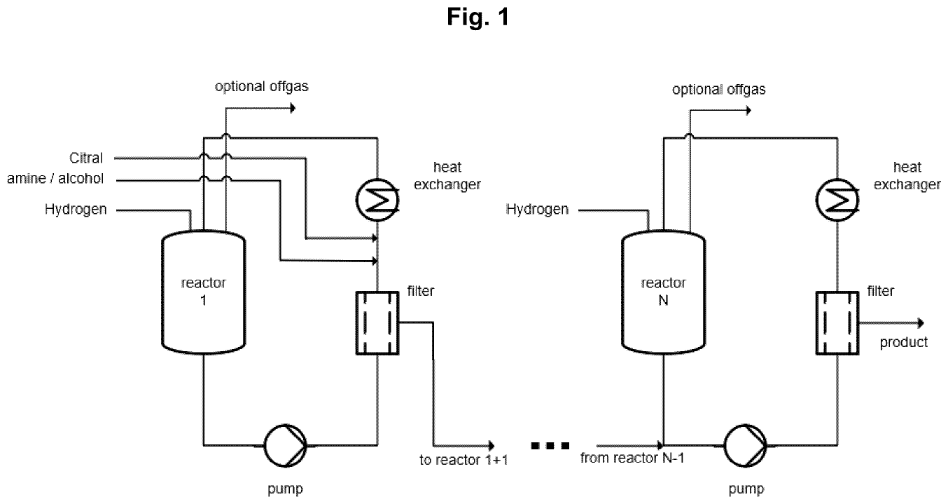

Typically, a reactor cascade shown in FIG. 1 is used consisting of N reactors (total number of reactors, which contain suspended catalyst), where N is >2. N is preferably 2 to 7, more preferred 2 to 5, most preferred 2, or 3. Citral, the amine and the alcohol are fed to the first reactor (reactor 1). Hydrogen is supplied for the hydrogenation and to adjust the desired pressure in the reaction system. Optionally, off-gas can be used to adjust the desired pressure and/or to purge volatile by-components. The temperature is adjusted using a heat exchanger which can either be integrated in the reactor or preferably be in the loop of the reactor. An external loop is established via a pump. A filter is used to keep the solid catalyst in the reaction system, the filter is preferably a membrane filter. The reactor can be, for example, a stirred vessel reactor, a bubble column reactor, or a jet loop reactor. The transfer of partly hydrogenated product and the alcohol/amine solution from one reactor to the next reactor is preferably done by pressure difference between the reactor loops, but can also be done via pump.

The final desired product concentration is adjusted in the last reactor, here the final product is released from the filter and can then be further proceeded, for example, purified by distillation, or directly used in the isomerization of the mixture of geraniol and nerol to linalool.

In the process of the present invention a cascade, i.e. series of at least two reactors is used. The process preferably takes place in a cascade of stirred vessel reactors, a cascade of bubble column reactors, or a cascade of jet loop reactors. The series of the at least two reactors may consist of two, or more different reactors, which are, for example, selected from a stirred vessel reactor, a bubble column reactor and a jet loop reactor.

More preferably, the reaction takes place in a cascade of jet loop reactors, wherein two to seven, especially two to five, very especially two, or three jet loop reactors are arranged in series; the reaction takes place in a cascade of stirred vessel reactors, wherein two to seven, especially two to five, very especially two, or three stirred vessel reactors are arranged in series.

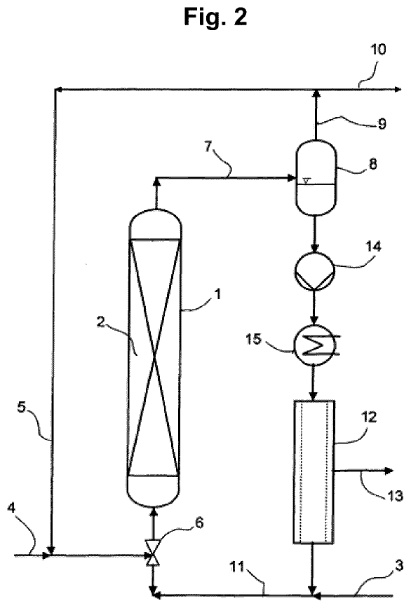

Bubble column reactors are, for example, described in EP1318128B1 and Ullmann's Encyclopedia of Industrial Chemistry, 2012 Wiley-VCH Verlag GmbH & Co. KGaA, Weinheim, chapter “Bubble Columns”. Reference is made to FIG. 2a.

FIG. 2 shows, for example, the experimental construction of a continuously operated reactor (bubble column) 1 with suspended catalyst 2, which can be fed in through the pipe 3. Fresh hydrogen gas is supplied by pipe 4. The circulation gas 5 is mixed by means of the mixing jet 6 with fresh gas and the suspension 11 that is circulated by means of the pump 14. The reactor effluent is transferred through the pipe 7 into the separating vessel 8 where the gas phase is separated off and passed out through pipe 9. In order to limit the buildup of gaseous impurities, part of this gas flow is discharged through the pipe 10 and the remainder is passed into the reactor through the pipe 5. The suspended catalyst remains in the reactor system by being retained in the filter 12, only catalyst-free liquid phase exiting through pipe 13 in the subsequent reactor. The temperature in the reactor system can be set as desired by means of the heat exchanger 15.

Jet loop reactors are, for example, described in Ullmann's Encyclopedia of Industrial Chemistry, 2017 Wiley-VCH Verlag GmbH & Co. KGaA, Weinheim, chapter “Loop Reactors”. A jet loop reactor in the sense of the present invention relates to a device for the continuous reaction of a liquid, in which the liquid under pressure passes through a nozzle into a reaction chamber inlet. This longitudinal flow through a main stream direction at the end of the reaction chamber located with respect to the nozzle is deflected, flows back against the main current direction and again in the main stream direction is accelerated, so that inside the reactor space an internal liquid circuit (loop) is established. Reference is made to FIG. 2b. Typically citral, the amine and the alcohol are fed to the reactor, which contains suspended catalyst. Hydrogen is supplied for the hydrogenation and to adjust the desired pressure in the reaction system. Optionally, off-gas can be used to adjust the desired pressure and/or to purge volatile by-components. The temperature is adjusted using a heat exchanger which can either be integrated in the reactor or preferably be in loop of the reactor. A filter is used to keep the solid catalyst in the reaction system, the filter is preferably a membrane filter. The transfer of partly hydrogenated product and the alcohol/amine solution from one reactor to the next reactor is preferably done by pressure difference between the reactor loops, but can also be done via pump.

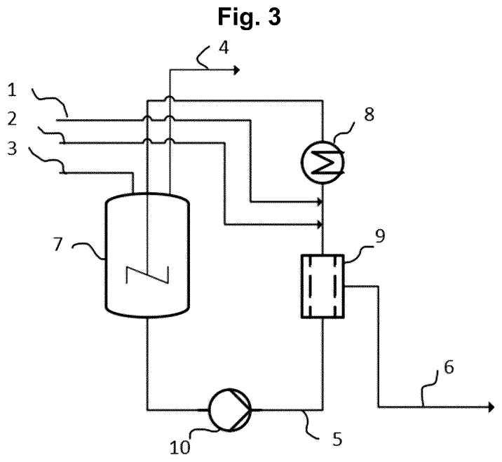

FIG. 3 shows, for example, the experimental construction of a continuously operated stirred vessel reactor 7 with suspended catalyst. Fresh hydrogen gas is supplied by pipe 3. Citral (pipe 1) and the amine as well as the alcohol (pipe 2) are fed into the reactor loop, alternatively they can also be fed directly into the stirred vessel reactor. An external loop (5) is established via pump 10. The temperature in the reactor is adjusted with the help of heat exchanger 8. An optional offgas (pipe 4) can be used to adjust the desired pressure in the reactor system and/or to purge any unwanted byproducts. The suspended catalyst remains in the reactor system by being retained in the filter 9, only catalyst-free liquid phase exiting through pipe 6 in the subsequent reactor.

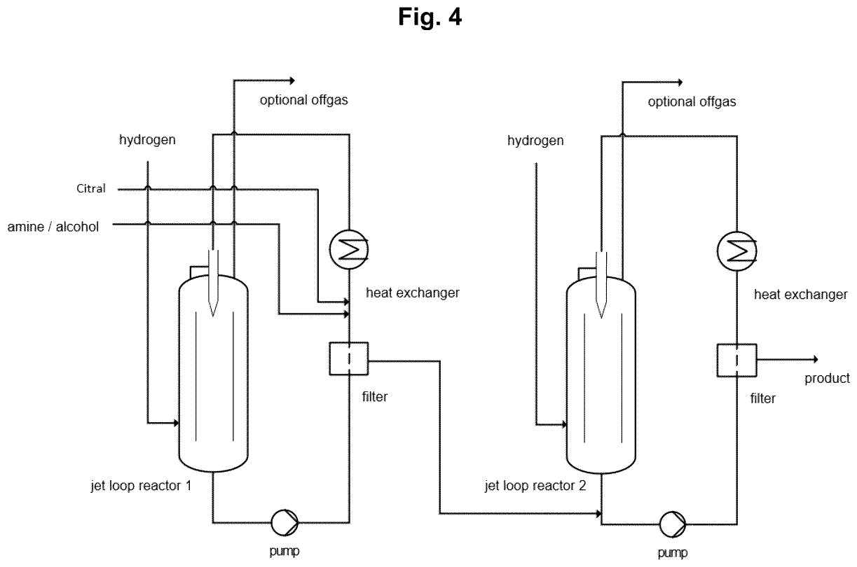

Most preferred, the hydrogenation of Citral is done using two successive, fully continuously operated loop reactors, i.e. a first (main)reactor 1 and a second (post)reactor 2. Reference is made to FIG. 4. The catalyzed reaction takes place in a two-stage reaction cascade under elevated hydrogen pressure. The catalyst suspension as an aminic alcoholic suspension is first placed in the reactors. The reaction device is now set to reaction conditions, i.e. in particular, it is brought to the desired temperature via heat exchanger, and the desired hydrogen partial pressure is set by pressurization of hydrogen. Citral as an aminic alcoholic solution is continuously supplied to the reaction device. Hydrogen is supplied to each individual reactor. The catalyst is retained by means of filtration in the respective reactor system. In the first (main)reactor, a conversion of Citral of >80% is achieved. The filtered reactor discharge from the first reactor is transferred to the second reactor. The subsequent reaction to almost full conversion (>98%) takes place in the 2^{nd} continuously operated loop reactor. The desired temperature is again adjusted by a heat exchanger and the desired hydrogen partial pressure by a hydrogen stream. The reaction mixture is continuously removed from the last reactor loop and processed.

The work-up of the reaction mixture obtained in the hydrogenation of the inventive process and the isolation of the product of the formula I are effected in a customary manner, for example by crystallization, an aqueous extractive work-up or by a distillative separation, for example under reduced pressure. The product of formula I may be obtained in sufficient purity by applying such measures or a combination thereof, obviating additional purification steps. Alternatively, further purification can be accomplished by methods commonly used in the art, such as chromatography and/distillation.

Preferably the work-up and the isolation of the product of the formula I are effected by removing volatile components, such as solvent and the tertiary amine, e.g. by distillation and finally isolating the product of formula I by vacuum rectification (see, for example, DE10223974). More preferably, the product of formula I is used without vacuum rectification in the production of linalool.

If Citral is used as starting material, a raw product is obtained after hydrogenation, which is a mixture of geraniol and nerol having a level of the by-product citronellol in total of less than 2.0% by weight, preferably less than 1.6% by weight, and in particular from 0.3 to 1.0% by weight, in particular a mixture of geraniol and nerol, which comprises 88 to 97% by weight, preferably 90 to 96% by weight, of geraniol and nerol; and 2.0 to 0.1% by weight, preferably 1.6 to 0.3% by weight, more preferably 1.0 to 0.3% by weight of the by-product citronellol, relative in each case to the total weight of the mixture excluding solvent and amine after reaction.

The process according to the present invention may comprise the additional step of isomerizing the mixture of geraniol and nerol to linalool in the presence of a dioxotungsten(VI) complex of the general formula

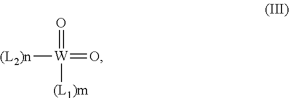

wherein L₁ and L₂ are independently of each other a ligand selected from the group consisting of the aminoalcohols, the aminophenols and mixtures thereof; and m and n are each 1 or 2. Reference is made to WO03/048091 and WO03/047749.

Additional Catalysts, which can be used in the isomerization, are, for example, described in CN105218312B and CN111087343B.

Accordingly, the mixture of geraniol and nerol obtained by the process according to the present invention may be used for the production of linalool.

Due to the low amount of the by-product citronellol the mixture of geraniol and nerol obtained by the process according to the present invention may advantageously be used directly for the production of linalool without further purification steps.

L₁ and L₂ are preferably 8-hydroxyquinoline. The ratio of tungsten to 8-hydroxyquinoline in the complex is between 1:1 and 1:5. The dioxotungsten(VI) complex of the formula (111) is preferably prepared prior to the reaction or in situ in the precursor allyl alcohol.

Linalool has a lower boiling point than geraniol and nerol, so that it may be removed from the equilibrium by distillation. This results in new linalool being constantly formed in the equilibrium reaction. The reaction is carried out at temperatures of 130-220° C., preferably 150-200° C., either at atmospheric pressure or under reduced pressure.

Hydrogenation of linalool in the presence of a suitable catalyst results in tetrahydrolinalool (3,7-dimethyloctan-3-ol) of formula

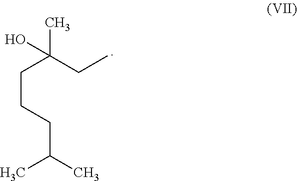

Reference is made to CN115925512A.

The catalyst is preferably a supported catalyst, the carrier of the supported catalyst is one or more selected from carbon, alumina, silica gel, diatomite and ZSM-5 and the active component is one or more selected from Ru, Pt, Pd, Ni, Rh and Ir.

The hydrogen pressure is 0.01 to 5.0 MPa, preferably 0.3 to 0.8 MPa, more preferably 0.4 to 0.5 MPa. The rection temperature is in the range of from 60 to 110° C., preferably 70 to 100° C., and more preferably 80 to 100° C.

If linalool of high purity is used as starting material tetrahydrolinalool of high purity is obtained,

The examples below serve to illustrate the invention without limiting it in any way.

### EXAMPLES

### Synthesis Example 1—Preparation of Catalyst B (in Analogy to WO2017060243A1, Example 1 D)

110 g of the activated carbon Norit SX Plus® were introduced without further pretreatment into a stirred flask with 2 L of water, suspended and heated to 80° C. under reflux. The pH was then raised to 9 by adding aqueous NaOH (1 mol/1). Within one hour, 300 ml. of a solution of ruthenium nitrosyl nitrate and iron nitrate (concentration corresponding to 5.85 g of Ru and 1.17 g of Fe) are added dropwise at 80° C. while at the same time maintaining the pH at about 9 by simultaneously adding aqueous NaOH. Stirring was continued at 80° C. for one hour and then the mixture was cooled. The cold suspension was filtered and washed with 40 L of water, then dried in a vacuum drying cabinet at 80° C. for 16 h. The dried powder was then reduced in a rotary sphere oven in a stream consisting of 70% hydrogen and 30% nitrogen at 200° C. for 3 h. After the end of the reduction, cooling was carried out under nitrogen and the catalyst was passivated with a gas mixture of 1% oxygen in nitrogen. The catalyst had a Ru content of 5.0% by weight, an Fe content of 1.0% by weight and an Na content of 0.036% by weight.

### Example 1

The reaction network and related kinetic parameters for the hydrogenation system of Citral to Geraniol/Nerol were determined based on multiple batch hydrogenation trials in an autoclave. With the help of the kinetic data simulation studies of reaction cascades of different sizes were performed.

In Example 1 a feed composition shown in Table 1, a hydrogenation pressure of 30 bar and a temperature of 90° C. were used. The number of cascaded reactors was two or three, where one reactor is the reference for a normal one-stage continuous reaction system.

TABLE 1

| Species | Amount | |
|---|---|---|
| Catalyst B | 15 | g/kg |
| Trimethylamine | 4.44% | by weight |
| Methanol | 20.25% | by weight |
| cis-Citral | 6.87% | by weight |
| trans-Citral | 63.5% | by weight |
| iso-Citrale | 4.27% | by weight |

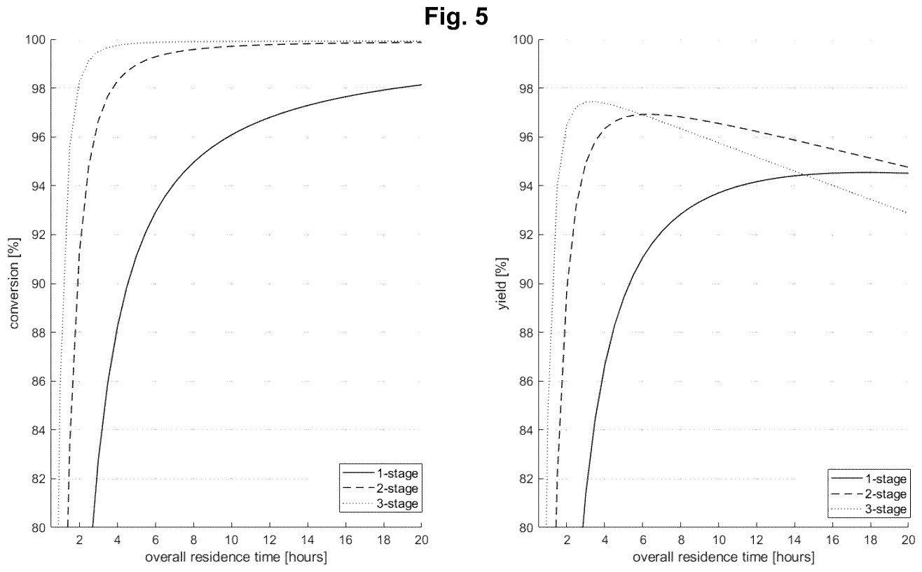

The results obtained in Example 1 are shown in Table 2 and FIG. 5. For the 2-stage cascade a maximum yield of 96.92% is achieved after around 6 hours total residence time and for the 3-stage cascade the maximum yield of 97.43% is achieved after around 3 hours total residence time. As reference the 1-stage process results in a maximum yield of 94.54% after 17 hours. Accordingly, the reactor cascade leads to higher yields (higher selectivity at high conversion rates) and also leads to a reduced overall residence time.

While the specific numbers given in Table 2 and FIG. 5 will depend on the hydrogenation pressure, temperature, catalyst amount, catalysat activity etc., a reactor cascade is always more favorable as compared to the 1-stage continuous process.

TABLE 2

| Residence | Conversion | | | Yield | | |
|---|---|---|---|---|---|---|
| Time | 1-stage | 2-stage | 3-stage | 1-stage | 2-stage | 3-stage |
| 1 | 44.34 | 66.59 | 85.78 | 43.68 | 65.68 | 84.53 |
| 2 | 70.86 | 91.18 | 98.23 | 69.77 | 89.74 | 96.5 |
| 3 | 82.74 | 96.64 | 99.48 | 81.39 | 94.93 | 97.43 |
| 4 | 88.19 | 98.28 | 99.75 | 86.65 | 96.34 | 97.38 |
| 5 | 91.12 | 98.95 | 99.83 | 89.42 | 96.8 | 97.17 |
| 6 | 92.9 | 99.28 | 99.87 | 91.06 | 96.92 | 96.9 |
| 7 | 94.1 | 99.46 | 99.89 | 92.12 | 96.9 | 96.62 |
| 8 | 94.95 | 99.58 | 99.9 | 92.84 | 96.82 | 96.34 |
| 9 | 95.58 | 99.65 | 99.9 | 93.34 | 96.69 | 96.05 |
| 10 | 96.08 | 99.71 | 99.91 | 93.71 | 96.55 | 95.76 |
| 11 | 96.47 | 99.75 | 99.91 | 93.97 | 96.39 | 95.47 |
| 12 | 96.79 | 99.77 | 99.91 | 94.17 | 96.22 | 95.17 |
| 13 | 97.06 | 99.8 | 99.91 | 94.31 | 96.05 | 94.88 |
| 14 | 97.28 | 99.81 | 99.91 | 94.41 | 95.87 | 94.59 |
| 15 | 97.48 | 99.83 | 99.91 | 94.47 | 95.69 | 94.3 |
| 16 | 97.64 | 99.84 | 99.92 | 94.51 | 95.5 | 94.01 |
| 17 | 97.79 | 99.85 | 99.92 | 94.54 | 95.32 | 93.73 |
| 18 | 97.92 | 99.86 | 99.92 | 94.54 | 95.13 | 93.44 |
| 20 | 98.03 | 99.86 | 99.92 | 94.53 | 94.94 | 93.15 |

The maximum achievable yield increases with increasing number of reactors, as the selectivity is higher at higher conversion rates for cascades with more reactors. The required overall residence time decreases with increasing number of reactors, which is caused by the higher reaction rates in the first reactors of the cascade compared to the slow reaction rate in one single stage reactor.

In case the residence time is too low there is too much Citral remaining. If the chosen residence time is too long too much Citronellol is formed and the achievable yield decreases again. Consequently, the operating parameters have to be carefully adjusted to achieve the desired yield and selectivity.

### Example 2

The mixture of geraniol and nerol obtained in Example 1 is isomerized in analogy to Example 3 of WO03/047749 to linalool. The isomerization may be accelerated by removing water with the aid of a gas stream.

1.-15. (canceled)

16. A process for the preparation of unsaturated alcohols of the formula 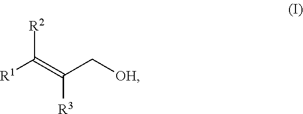
wherein
R¹ and R² are each independently from one another selected from C₁-C₂₀-alkyl which is unsubstituted or substituted with 1 to 5 identical or different radicals R⁴, C₂-C₂₀-alkenyl which contains 1, 2, 3, 4 or 5 double bonds and is unsubstituted or substituted with 1 to 5 identical or different radicals R⁵; provided that R¹ and R² are different from each other;
R³ is selected from hydrogen and C₁-C₄-alkyl;
R⁴ is selected from hydroxyl, cyano, C₃-C₇-cycloalkyl and C₁-C₄-alkoxy,
R⁵ is selected from hydroxyl, cyano, C₁-C₄-alkyl, C₃-C₇-cycloalkyl, C₁-C₄-alkoxy; which comprises subjecting unsaturated aldehydes of the formula 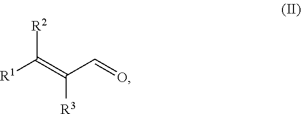
wherein R¹, R² and R³ have the above defined meanings, to a hydrogenation in the presence of a catalyst and a tertiary amine; characterized in that the hydrogenation takes place in a reactor cascade.

17. The process as claimed in claim 16, wherein one of R¹ and R² is C₄-C₁₀-alkenyl containing one double bond and the other one is methyl or ethyl; and R³ is hydrogen.

18. The process as claimed in claim 16, wherein the unsaturated aldehyde of the formula (II) is citral, which is hydrogenated to give a mixture of geraniol and nerol.

19. The process according to claim 18, wherein the citral is used in the process as an aminic alcoholic solution.

20. The process according to claim 16, wherein the concentration of citral in the solution is in the range from 50 to 90% by weight based on the total amount of citral, alcohol and amine.

21. The process according to claim 16, wherein the concentration of alcohol in the solution is in the range from 40 to 5% by weight based on the total amount of citral, alcohol and amine.

22. The process according to claim 16, wherein the concentration of amine in the solution is in the range from 1 to 15% by weight, or 0.001 to 0.7% by weight, based on the total amount of citral, alcohol and amine.

23. The process according to claim 16, wherein the amine is a tertiary amine.

24. The process according to claim 23, wherein the tertiary amine is a tri(C₁-C₂₀-alkyl)amine.

25. The process according to claim 16, wherein the catalyst is a heterogeneous catalyst, which is a supported catalyst which comprises ruthenium and additionally iron as supported on an inert support material.

26. The process according to claim 18, wherein the reaction takes place in a cascade of stirred vessel reactors, a cascade of bubble column reactors, or a cascade of jet loop reactors.

27. The process according to claim 26, wherein the reaction takes place in a cascade of jet loop reactors, wherein two to seven jet loop reactors are arranged in series; or the reaction takes place in a cascade of stirred vessel reactors are arranged in series.

28. The process according to claim 18, wherein the product obtained after hydrogenation is a mixture of geraniol and nerol having a level of the by-product citronellol in total of less than 2.0% by weight.

29. The process according to claim 28, wherein the product obtained after hydrogenation is a mixture of geraniol and nerol, which comprises 88 to 97% by weight of geraniol and nerol; and 2.0 to 0.1% by weight of the by-product citronellol, relative in each case to the total weight of the mixture excluding solvent and amine after reaction.

30. The process according to claim 27, which comprises isomerizing the mixture of geraniol and nerol to linalool in the presence of a dioxotungsten(VI) complex of the general formula 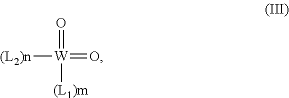
wherein L₁ and L₂ are independently of each other a ligand selected from the group consisting of aminoalcohols, aminophenols and mixtures thereof; and m and n are each 1 or 2.

31. A method for the production of linalool, the method comprising using the mixture of geraniol and nerol obtained by the process according to claim 28.
